(19) United States
(12) Patent Application Publication
(10) Pub. No.: US 2024/0374841 A1
(43) Pub. Date: Nov. 14, 2024

(54) Activation sleeve and subassembly for a medicament delivery device

(71) Applicant: SHL Medical AG, Zug (CH)

(72) Inventors: Daniel Carlsson, Nacka Strand (SE); Daniel Säll, Nacka Strand (SE)

(21) Appl. No.: 18/687,964

(22) PCT Filed: Aug. 18, 2022
(86) PCT No.: PCT/EP2022/073055

§ 371 Date: Feb. 29, 2024

(30) Foreign Application Priority Data
Sep. 1, 2021 (EP) 21194410.3

(51) Int. Cl. A61M 5/32; A61M 5/20; A61M 5/315

(52) U.S. Cl. CPC A61M 5/3245; A61M 5/2033; A61M 5/315; A61M 2005/2073; A61M 2205/276

(57) ABSTRACT

The present disclosure provides an activation sleeve for a medicament delivery device, configured to operatively connect a needle cover and an activation button of a medicament delivery device. The activation sleeve comprises distal part and a proximal part coaxially connected to the distal part. The proximal part comprises a needle cover contact surface configured to contact the needle cover of the medicament delivery device, whereby the activation sleeve is distally movable in accordance with a distal movement of the needle cover inside a proximal housing of the medicament delivery device. The distal part comprises an inclined activation profile on its outer circumferential surface configured to interact with the activation button of the medicament delivery device. The inclined activation profile is configured to rotate the activation sleeve due to its interaction with the activation button when the activation button and the activation sleeve are moved towards each other in any order. Subassembly comprising said activation sleeve and medicament delivery device comprising the subassembly or the activation sleeve.

### CROSS-REFERENCE TO RELATED APPLICATIONS

The present application is a U.S. National Phase Application pursuant to 35 U.S.C. § 371 of International Application No. PCT/EP2022/073055 filed Aug. 18, 2022, which claims priority to European Patent Application No. 21194410.3 filed Sep. 1, 2021. The entire disclosure contents of these applications are herewith incorporated by reference into the present application.

### TECHNICAL FIELD

The present invention relates to an activation sleeve for a medicament delivery device which enables a sequence independent activation of the medicament delivery device. Further, the present invention relates to a subassembly for a medicament delivery device comprising said activation sleeve. Further, the present invention relates to a medicament delivery device comprising said subassembly or said activation sleeve, so that the medicament delivery device can be activated in a sequence independent manner.

### RELATED ART

Medicament delivery devices, especially disposable single-dose auto-injectors, are often intended to be used by users in a home environment, enabling the user to self-apply a medical substance, for example insulin or hormones, without a medically trained person being present. Resulting from that, easy and safe handling of such devices is a key design aspect. Thus, depending on the intended use and type of medicament, medicament delivery devices having a varying degree of automatic functions have been developed to facilitate injection of medicaments in a reliable and safe manner.

Auto-injectors with a penetration function usually comprise a housing, a container holder carrying a medicament container with a needle and a plunger rod as well as a compression spring providing a force to move the plunger rod with respect to the housing and/or container holder. Such auto-injectors often also comprise a needle cover and an end cap protecting the tip of the needle in order to prevent injuries and contamination of the needle, when the injector is not used and/or after the injection is completed. Moreover, existing auto-injectors usually comprise an activation button that enables the user to determine the time of injection.

When said auto-injectors are actuated, the container holder and the plunger rod, which are slidable in the housing, are jointly driven towards the user's skin by the force of the compression spring, which causes the needle to penetrate the user's skin. After having reached a predetermined position, the plunger rod is moved towards the user's skin, thereby acting on a stopper inside the medicament container such that the medicament is expelled through the needle.

Some auto-injectors may further have an automatic safety function for preventing accidental needle sticks by covering the needle with the needle cover when the injector is withdrawn from the user's skin.

Prior to the actuation, the auto-injector is in a secured state, in which the end cap is attached to the housing and a movement of the plunger rod is inhibited. Initiating an injection usually requires the user to activate the injector by consecutively performing three steps. First, the user needs to remove the end cap from the housing. After that, the user needs to press the needle cover against the intended injection site with a predetermined force. Third, the user needs to press the activation button which initiates the actuation.

Such a the three-step activation as used in conventional auto-injectors and provides sufficient safety when it comes to avoiding injuries and premature medicament discharge, for example when the injector is dropped to the floor. However, this activation process is prone to error as the above-mentioned user interactions must be performed in the correct sequence. Otherwise, the auto-injector might block, and the activation button cannot be pressed. This makes an injection of the medicament impossible.

Not performing the activation steps in the correct sequence is one of, if not the most common operating error.

Hence, it is an object of the present invention to simplify the activation process of a medicament delivery device, especially a disposable single-dose auto-injector, while still preventing injuries or accidental medicament discharge. Further, it is an object of the present invention to enable a sequence independent activation of a medicament delivery device.

### SUMMARY

The above-mentioned objects are achieved, and the problems are overcome by the activation member according to independent claim 1, the subassembly according to independent claim 9 and the medicament delivery device according to independent claim 15.

Further aspects, improvements and variations are disclosed in the dependent claims, the figures and the description.

In the present application, when the term “distal” is used, this refers to the direction pointing away from the injection site, i.e. the point at which the needle penetrates the user's body in order to inject the medicament. When the term “distal part/end” is used, this refers to the part/end of the delivery device, or the parts/ends of the members thereof, which is/are located furthest away from the injection site. Correspondingly, when the term “proximal” is used, this refers to the direction pointing to the injection site. When the term “proximal part/end” is used, this refers to the part/end of the delivery device, or the parts/ends of the members thereof, which is/are located closest to the injection site.

The activation sleeve for a medicament delivery device, according to an embodiment of the present disclosure, is configured to operatively connect a needle cover and an activation button of the medicament delivery device. The activation sleeve extends along a longitudinal axis from a proximal to a distal end of the medicament delivery device. The activation sleeve comprises a needle cover contact surface configured to contact the needle cover of the medicament delivery device. The activation sleeve is distally movable in accordance with a distal movement of the needle cover inside a proximal housing of the medicament delivery device. The activation sleeve further comprises an inclined activation profile on its outer circumferential surface configured to interact with the activation button of the medicament delivery device. The inclined activation profile is configured to rotate the activation sleeve due to its interaction with the activation button, when the activation button and the activation sleeve are moved towards each other in any order.

Preferably, the activation sleeve comprises two inclined activation profiles on its outer circumferential surface configured to interact with the activation button of the medicament delivery device. Preferably, the two inclined activation profiles are positioned with an offset between 90° and 270° in a circumferential direction of the activation sleeve. More preferably, the two inclined activation profiles are positioned with an offset of 180° in a circumferential direction of the activation sleeve. Preferably, the distal part of the activation sleeve comprises the two inclined activation profiles on its outer circumferential surface.

Preferably, the activation sleeve further comprises at its distal part at least one opening on its circumferential surface, which is configured to match a geometry of a plunger rod blocking element of a support member of the medicament delivery device, which is coaxially connected to a distal housing of the medicament delivery device. The at least one opening provides a space into which the plunger rod blocking element can deflect.

Preferably, the activation sleeve comprises two of the before described openings. Preferably, the distal part of the activation sleeve comprises the at least one opening or the two openings.

Preferably, the activation sleeve further comprises a first rotation lock profile configured to engage with a first anti-rotation member of the support member for blocking a rotation of the activation sleeve in a non-activated state, i.e. a state prior to the distal movement of the needle cover inside the proximal housing of the medicament delivery device. Preferably, the first rotation lock profile is configured to disengage from the first anti-rotation member when the activation sleeve is moved distally along a predetermined distance. Due to this distal movement, the activation sleeve is free to rotate.

Preferably, the distal part of the activation member comprises the first rotation lock profile.

Preferably, the inclined activation profile is configured to interact with a corresponding inclined activation button profile, when the activation button is pressed and thereby moved proximally in relation to the housing. This causes the activation sleeve to rotate until each of the at least one opening overlaps with the geometry of the plunger rod blocking element of the support member in the circumferential direction. Thus, the plunger rod blocking element can move radially outwardly in order to disengage from the plunger rod, when an axial force is applied to the plunger rod.

Preferably, the inclination of the inclined activation profile and the corresponding inclined activation button profile are the same.

Preferably, the activation sleeve further comprises a support ledge configured to support a proximal end of a distal spring of the medicament delivery device. Preferably, the needle cover contact surface is formed by a proximal surface of the support ledge.

Preferably, the first rotation lock profile comprises a protrusion which protrudes proximally from the support ledge. Preferably, at least a portion of the proximal end surface of the first rotation lock profile is inclined in a circumferential direction, in order to facilitate the assembly of the medicament delivery device.

Preferably, the activation sleeve comprises a second rotation lock profile configured to engage with a second anti-rotation member of the support member. Preferably, the distal part of the activation sleeve comprises the second rotation lock profile. Preferably, the second rotation lock profile has a similar geometry as the first rotation lock profile.

Preferably, the second rotation lock profile is positioned with an offset between 90° and 270° in a circumferential direction of the activation sleeve with respect to the first rotation lock profile. More preferably, the second rotation lock profile is positioned with an offset of 180° in a circumferential direction with respect to the first rotation lock profile.

Preferably, the activation sleeve comprises a proximal part.

Preferably, the proximal part is coaxially connected to the distal part.

Further preferably, the proximal part comprises the needle cover contact surface.

It is further preferred that the distal part comprises the inclined activation profile on its outer circumferential surface.

Preferably, the proximal part comprises at least one arm extending along the longitudinal axis. More preferably, the proximal part comprises two arms extending along the longitudinal axis. Preferably, the needle cover contact surface is formed by the proximal end surface of the at least one arm.

Preferably, each of the at least one arm has a support rib that, at least partially, extends along its length. Preferably the support rib protrudes in a radial inward direction. The support rib increases the mechanical stability of the arm in each spatial direction.

Preferably, when there are two arms, the two arms are positioned with an offset between 90° and 270° in a circumferential direction of the activation sleeve. More preferably, the two arms are positioned with an offset of 180° in a circumferential direction of the activation sleeve.

Preferably, when there are more than two arms, the arms are distributed evenly in a circumferential direction.

The subassembly for a medicament delivery device, according to an embodiment of the present disclosure, comprises a housing and a drive mechanism. The drive mechanism comprises a drive spring and a plunger rod configured to act on a medicament container placed in the medicament delivery device, for discharging a medicament therefrom. The subassembly further comprises a distal end part comprising an activation button which is displaceable in relation to the housing and a support member coaxially connected to the housing. The support member comprises a plunger rod blocking element configured to interact with the drive mechanism. The subassembly further comprises an activation sleeve according to one or more of the foregoing paragraphs or embodiments, respectively. The subassembly further comprises a distal spring arranged between the activation button and the activation sleeve and configured to apply a biasing force on the activation sleeve. The drive mechanism is in a pre-tensioned and locked state, when the medicament delivery device is in the non-activated state. The activation sleeve is configured to rotate due to an interaction of the inclined activation profile with the activation button when the activation button and the activation sleeve are moved towards each other in any order.

Preferably, the subassembly is a distal subassembly for a medicament delivery device, and so the housing may be named a distal housing. This applies to all other components of the subassembly, likewise.

Preferably, the support member is formed integrally with the housing. Alternatively, the support member is fixedly mounted to the housing, for example via at least one connection element.

Preferably, the housing comprises an activation button stop surface, which is configured to engage with a proximal end surface of the activation button in order to stop a proximal movement of the activation button into the housing at a predetermined position. Preferably, the activation button stop surface is formed by at least one radial inward protrusion on the inner wall of the housing. Alternatively, the activation button stop surface is formed by the connection element.

Preferably, the plunger rod blocking element is configured to releasably engage with a distal ledge of the plunger rod, such that a proximal movement of the plunger rod is blocked when the medicament delivery device is in the non-activated state. More preferably, the plunger rod blocking element comprises at least one support arm configured to releasably engage with a distal ledge of the plunger rod, such that a proximal movement of the plunger rod is blocked when the medicament delivery device is in the non-activated state.

Preferably, due to the rotation of the activation sleeve, each of the at least one openings of the activation sleeve is rotated to a position in which it matches the circumferential geometry of each of the at least one support arm of the support member. Thus, a radial outward deflection of each of the at least one support arm is enabled in order to disengage each of the at least one support arm from the distal ledge of the plunger rod and activate the medicament delivery device.

Preferably, each of the support arm is configured to interact with the distal ledge of the plunger rod via a radially inwardly inclined surface that is inclined in a proximal direction, such that due to the force of the pre-tensioned drive spring, the distal ledge of the plunger rod deflects each of the at least one support arm radially outwardly, in order to unlock the plunger rod and activate the medicament delivery device. Preferably, the radially inwardly inclined surface has a gradual inclination in a proximal direction.

Preferably, the activation button comprises a guide hole along a longitudinal axis of the subassembly configured to axially guide a distal end of the plunger rod when the medicament delivery device is activated.

The medicament delivery device, according to an embodiment of the present disclosure, comprises a distal subassembly and a proximal subassembly. The distal subassembly is a subassembly according to one or more of the foregoing paragraphs or embodiments, respectively.

Preferably, the medicament delivery device is a single-dose disposable auto-injector.

Preferably, the proximal subassembly comprises a proximal housing connectable to the distal housing of the distal subassembly. Further preferably, the proximal subassembly comprises a rotator coaxially connected to a distal inner part of the proximal housing. Preferably, the rotator is movable in relation to the proximal housing. Preferably, the proximal subassembly further comprises a displaceable needle cover partially arranged within the proximal housing and a medicament container holder connected through the needle cover and the rotator to the proximal housing. Preferably, the medicament container holder is configured to receive a medicament container.

Preferably, the proximal subassembly further comprises a needle cover spring disposed between the needle cover and the medicament container holder. Preferably, the needle cover spring is configured to apply a biasing force on the needle cover. Preferably, the needle cover is axially movable inside at least the proximal housing such that the distal end of the needle cover contacts the contact surface of the activation sleeve whereby the activation sleeve is distally displaced.

The activation sleeve and subassembly according to embodiments of the present disclosure enables a sequence independent activation of a medicament delivery device in which it operatively connects the activation button with the needle cover. In particular, with the activation sleeve according to the present disclosure, it is irrelevant whether the needle cover is pushed distally before the activation button is pressed, or if the activation button is pressed before. Thus, a major operating error which can block the medicament delivery device or avoid an injection, is eliminated. This increases the safety and reliability of the medicament delivery device, thereby improving usability.

### BRIEF DESCRIPTION OF THE DRAWINGS

The following Figures below disclose an embodiment of the invention for illustrational purposes only. In particular, the disclosure within the Figures is not meant to limit the range of protection of the invention. The embodiment shown may be modified in many ways within the scope of the claims.

FIG. 1 shows a perspective view of an activation sleeve according to a preferred embodiment of the invention;

FIG. 2 shows a perspective view of an activation sleeve according to another preferred embodiment of the invention;

FIG. 3A shows a partially cutaway perspective view of a subassembly according to a preferred embodiment of the invention;

FIG. 3B shows a cross-sectional view of the subassembly of FIG. 3A;

FIG. 3C shows a zoomed-in cross-sectional view of the plunger rod support of the subassembly according to FIGS. 3A and 3B;

FIG. 4 shows a cross-sectional view of the activation button according to an embodiment of the invention;

FIG. 5 shows a perspective view of a medicament delivery device according to a preferred embodiment of the invention;

FIG. 6 shows a perspective view of a proximal subassembly of the medicament delivery device of FIG. 5; and

FIG. 7 shows a perspective view of a needle cover according to another embodiment of the invention.

### DETAILED DESCRIPTION

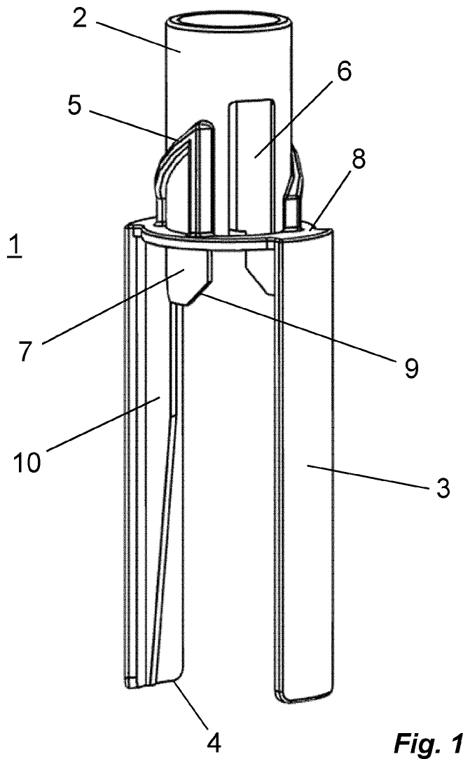

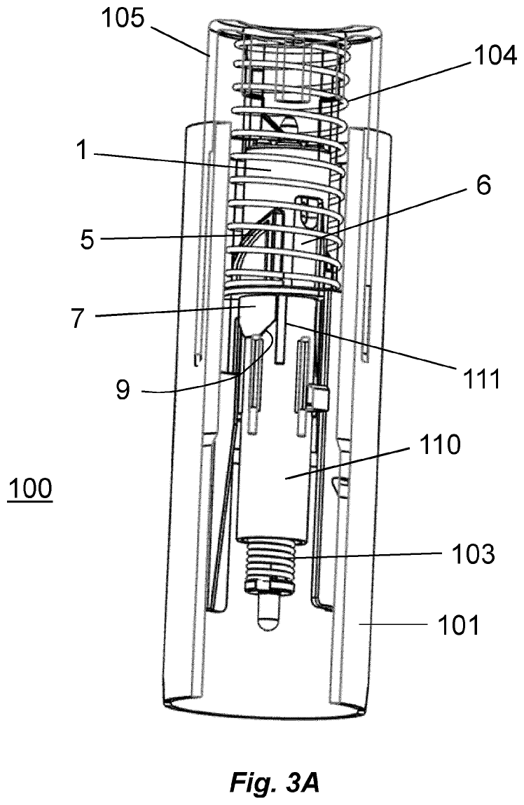

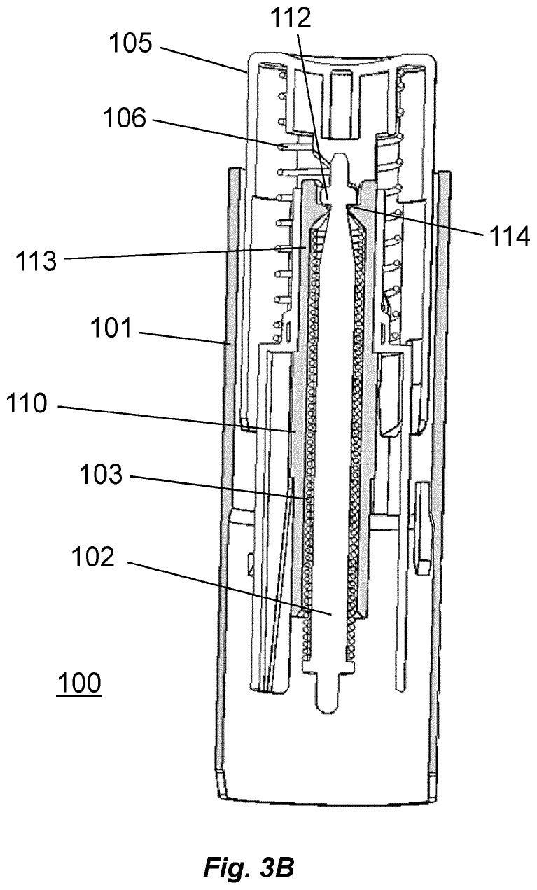

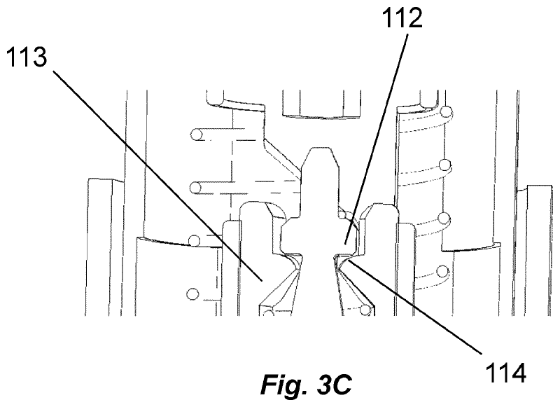

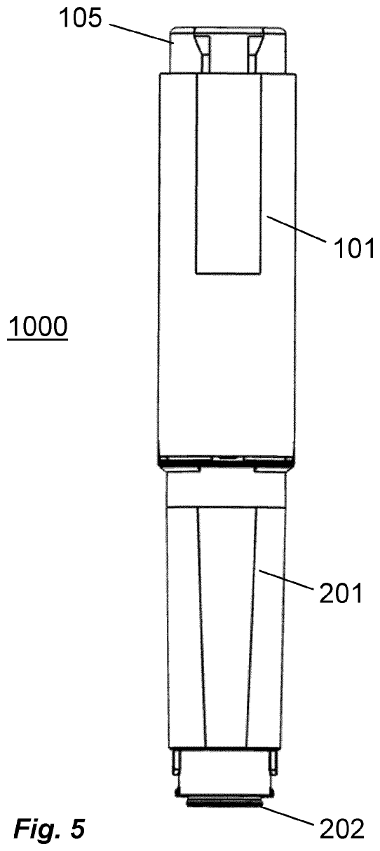

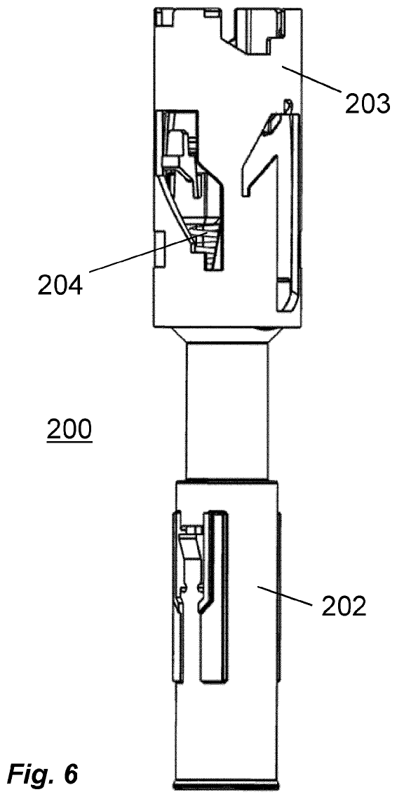

FIG. 1 shows a perspective view of an activation sleeve 1 for a medicament delivery device according to a preferred embodiment of the present disclosure. The activation sleeve 1 is configured to operatively connect a needle cover 202 (see FIGS. 5 and 6) of a medicament delivery device to and an activation button 105 (cf. FIGS. 3A, 3B and 5) of the medicament delivery device. The activation sleeve 1 extends along a longitudinal axis from a proximal to a distal end of the medicament delivery device. The activation sleeve 1 comprises a distal part 2 and a proximal part 3 coaxially connected to the distal part 2. The proximal part 3 comprises a needle cover contact surface 4 configured to contact the needle cover of the medicament delivery device (cf. FIGS. 5-7). The activation sleeve 1 is distally movable in accordance with a distal movement of the needle cover 202 inside a proximal housing 201 (cf. FIG. 5) of the medicament delivery device. The distal part 2 comprises an inclined activation profile 5 on its outer circumferential surface configured to interact with the activation button 105 of the medicament delivery device. The inclined activation profile 5 is configured to rotate the activation sleeve 1 due to its interaction with the activation button 105, when the activation button 105 and the activation sleeve 1 are moved towards each other in any order.

According to an embodiment, the distal part 2 further comprises at least one opening 6 on its circumferential surface, which is configured to match a geometry of a plunger rod blocking element of a support member 110 (cf. FIGS. 3A and 3B) of the medicament delivery device. The opening 6 provides a space into which the plunger rod blocking elements can radially outwardly deflect.

According to an embodiment, the distal part 2 further comprises a first rotation lock profile 7 configured to engage with a first anti-rotation member 111 (cf. FIG. 3A) of the support member 110 for blocking a rotation of the activation sleeve 1 in a non-activated state, i.e. a state prior to the distal movement of the needle cover 202 inside the proximal housing 201 of the medicament delivery device. According to an embodiment, the first rotation lock profile 7 is configured to disengage from the first anti-rotation member 111 when the activation sleeve 1 is moved distally along a predetermined distance.

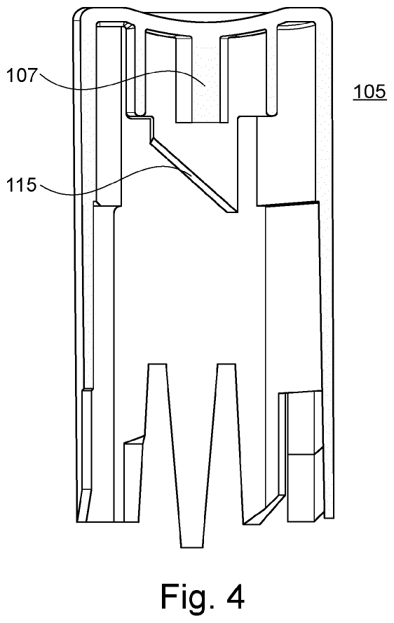

According to an embodiment, the inclined activation profile 5 is configured to interact with a corresponding inclined activation button profile 115 (cf. FIG. 4) of the activation button 105, when the activation button 105 is pressed and thereby moved proximally. This interaction causes the activation sleeve 1 to rotate, such that the at least one opening 6 overlaps with the plunger rod blocking element of the support member 110.

According to an embodiment, the inclination of the inclined activation profile 5 and the corresponding inclined activation button profile 115 are the same.

According to an embodiment, the activation sleeve 1 as shown in FIG. 1 further comprises a support ledge 8 configured to support a proximal end of a distal spring 104 (cf. FIG. 3A) of the medicament delivery device.

According to an embodiment, the first rotation lock profile 7 is a protrusion which protrudes proximally from the support ledge 8. Preferably at least a portion 9 of the proximal end surface of the first rotation lock profile 7 is inclined in a circumferential direction.

According to an embodiment, the proximal part 3 comprises two arms extending along the longitudinal axis. Preferably, the needle cover contact surface 4 is formed by the proximal end surface of the two arms.

According to an embodiment, each of the arms has a support rib 10 that, at least partially, extends along its length. According to an embodiment, the support rib 10 protrudes in a radial inward direction. The support rib 10 increases the mechanical stability of the arms in each spatial direction.

As shown the two arms are positioned with an offset of 180° in a circumferential direction of the activation sleeve 1.

According to an embodiment, the distal part 2 comprises a second rotation lock profile, which is configured to engage with a second anti-rotation member (not shown in FIG. 3A) of the support member 110. According to an embodiment, the second rotation lock profile has the same geometry as the first rotation lock profile 7.

According to an embodiment, the second rotation lock profile is positioned with an offset of 180° in a circumferential direction with respect to the first rotation lock profile 7.

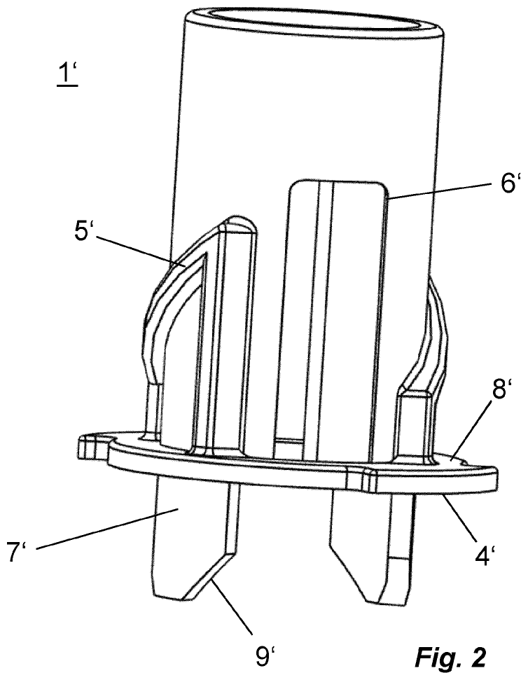

FIG. 2 shows another preferred embodiment of the activation sleeve 1′ according to the present disclosure. The activation sleeve 1′ has no proximal part. The other components of the activation sleeve 1′ correspond to the components of the activation sleeve 1 of FIG. 1. The respective reference signs of the components of the activation sleeve 1′ include an apostrophe.

According to an embodiment, the needle cover contact surface 4′ is formed by the support ledge 8′ configured to support a proximal end of a distal spring 104 (cf. FIG. 3A) of the medicament delivery device.

FIGS. 3A and 3B show the subassembly 100 for a medicament delivery device, according to a preferred embodiment of the present disclosure. The subassembly 100 comprises a housing 101 and a drive mechanism. The drive mechanism comprises a drive spring 103 and a plunger rod 102 configured to act on a medicament container placed in the medicament delivery device, for discharging a medicament therefrom. The subassembly 100 further comprises a distal end part comprising an activation button 105 which is displaceable in relation to the housing 101 and a support member 110 coaxially connected to the housing 101. The support member 110 comprises a plunger rod blocking element configured to interact with the drive mechanism. The subassembly 100 further comprises an activation sleeve 1 according to one or more of the foregoing paragraphs or embodiments, respectively. The subassembly 100 further comprises a distal spring 104 arranged between the activation button 105 and the activation sleeve 1 and configured to apply a biasing force on the activation sleeve 1. The drive mechanism is in a pre-tensioned and locked state, when the medicament delivery device is in a non-activated state. The activation sleeve 1 is configured to rotate due to an interaction of the inclined activation profile 5 with the activation button profile 115 (cf. FIG. 4) when the activation button 105 and the activation sleeve 1 are moved towards each other in any order.

According to an embodiment, as shown in FIG. 3B, the plunger rod blocking element comprises two supports arms 113 which are configured to releasably engage with a distal ledge 112 of the plunger rod 102, such that a proximal movement of the plunger rod 102 is blocked when the medicament delivery device is in the non-activated state.

According to an embodiment, due to the rotation of the activation sleeve 1, each of the at least one opening 6 of the activation sleeve 1 is rotated to a position in which it matches the circumferential geometry of each of the two support arms 113 of the support member 110. Thus, a radial outward deflection of the two support arms 113 is enabled in order to disengage each of the support arms 113 from the distal ledge 112 of the plunger rod 103 and activate the medicament delivery device.

According to an embodiment, the support arms 113 are configured to interact with the distal ledge 112 of the plunger rod 102 via a radially inwardly inclined surface 114 that is inclined in a proximal direction, such that due to the force of the pre-tensioned drive spring 103, the distal ledge 112 of the plunger rod 102 deflects each of the support arms 113 radially outwardly, in order to unlock the plunger rod 102 and activate the medicament delivery device. According to an embodiment, the radially inwardly inclined surface 114 has a gradual inclination in a proximal direction.

According to an embodiment, the activation button 105 as shown in FIG. 4 comprises a guide hole 107 along a longitudinal axis of the subassembly 100 configured to axially guide a distal end of the plunger rod 102 when the medicament delivery device is activated.

The medicament delivery device 1000 according to a preferred embodiment of the present disclosure, as shown in FIG. 5, comprises a distal subassembly and a proximal subassembly 200 (see FIG. 6). The distal subassembly is a subassembly 100 according to one of the embodiments described in the foregoing paragraphs.

According to an embodiment, the proximal subassembly 200 comprises a proximal housing 201 connectable to the distal housing 101 of the distal subassembly 100 and a displaceable needle cover 202 partially arranged within the proximal housing 201.

According to an embodiment shown in FIG. 6, the proximal subassembly 200 further comprises a rotator 203 coaxially connected to a distal inner part of the proximal housing 201 (not shown in

FIG. 6). The rotator 203 is movable in relation to the proximal housing 201. According to an embodiment, the proximal subassembly 200 further comprises a medicament container holder (not visible in FIGS. 5 and 6) connected through the needle cover 202 and the rotator 203 to the proximal housing 201.

According to an embodiment, the proximal subassembly 200 further comprises a needle cover spring 204 disposed between the needle cover 202 and the medicament container holder and configured to apply a biasing force on the needle cover 202. The needle cover 202 is axially movable inside at least the proximal housing 201 such that the distal end of the needle cover 202 contacts the needle cover contact surface 4 of the activation sleeve 1, when the needle cover is moved distally inside the housing 201. Due to the contact, the activation sleeve 1 is moved distally, as well.

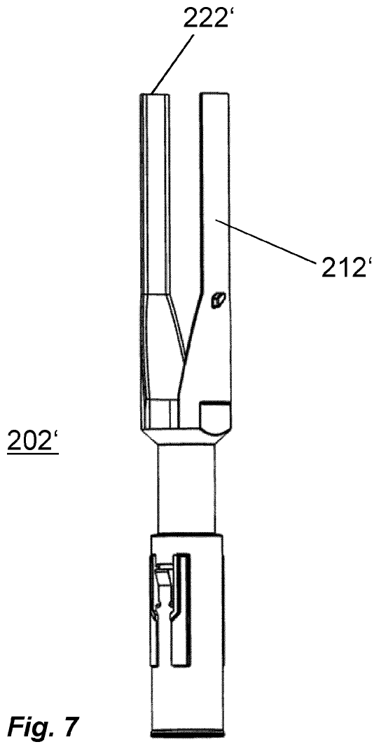

According to another embodiment, as shown in FIG. 7, the needle cover 202′ comprises two arms 212′ which extend in a distal direction along a longitudinal axis of the medicament delivery device 1000. The distal end surfaces 222′ of the two arms 212′ contact the needle cover contact surface 4′ of the activation sleeve 1′, when the needle cover 202′ is moved distally inside the proximal housing 201.

While the invention has been illustrated and described in detail in the drawings and the foregoing description, such illustration and description are to be considered illustrative or exemplary and not restrictive. It should be noted that the invention covers all further features shown in the figures individually although they may not have been described in the afore description and it will be understood that changes and modifications may be made by those of ordinary skill within the scope of the following claims. In particular, the present invention covers further embodiments with any combination of features from different embodiments described above and below.

Furthermore, in the claims the word “comprising” does not exclude other elements or steps, and the terms “a”, “an” “first”, “second” etc. do not exclude a plurality. A single unit may fulfil the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

1-16. (canceled)

17. An activation sleeve for a medicament delivery device, configured to operatively connect a needle cover and an activation button of a medicament delivery device, wherein the activation sleeve extends along a longitudinal axis from a proximal to a distal end, the activation sleeve comprising:
a needle cover contact surface configured to contact the needle cover of the medicament delivery device, whereby the activation sleeve is distally movable in accordance with a distal movement of the needle cover inside a proximal housing of the medicament delivery device; and
an inclined activation profile on its outer circumferential surface configured to interact with the activation button of the medicament delivery device,
wherein the inclined activation profile is configured to rotate the activation sleeve due to its interaction with the activation button when the activation button and the activation sleeve are moved towards each other in any order.

18. The activation sleeve of claim 17, wherein a distal part thereof comprises at least one opening on its circumferential surface, wherein each of the at least one opening is configured to match a geometry of a plunger rod blocking element of a support member which is coaxially connected to a distal housing of the medicament delivery device, in order to provide a space into which the plunger rod blocking element can deflect.

19. The activation sleeve of claim 17, wherein the distal part further comprises a first rotation lock profile configured to engage with a first anti-rotation member of the support member for blocking a rotation of the activation sleeve in a non-activated state, and wherein the first rotation lock profile is configured to disengage from the first anti-rotation member when the activation sleeve is moved distally along a predetermined distance.

20. The activation sleeve of claim 17, further comprising a support ledge configured to support a proximal end of a distal spring of the medicament delivery device.

21. The activation sleeve of claim 17, comprising a proximal part,
wherein the proximal part is coaxially connected to the distal part,
wherein the proximal part comprises the needle cover contact surface, and
wherein the distal part comprises the inclined activation profile on its outer circumferential surface.

22. The activation sleeve of claim 17, wherein the proximal part comprises at least one arm, preferably two arms, extending along the longitudinal axis.

23. The activation sleeve of claim 22, wherein each arm has a support rib that, at least partially, extends along its length, and wherein the support rib protrudes in a radial inward direction.

24. The activation sleeve of claim 17, wherein the distal part comprises a second rotation lock profile configured to engage with a second anti-rotation member of the support member.

25. A subassembly for a medicament delivery device comprising:
a housing;
a drive mechanism comprising a drive spring and a plunger rod configured to act on a medicament container placed in the medicament delivery device for discharging a medicament therefrom;
a distal end part comprising an activation button which is displaceable in relation to the housing;
a support member coaxially connected to the housing, wherein the support member comprises a plunger rod blocking element configured to interact with the drive mechanism,
an activation sleeve according to any one of the preceding claims; and
a distal spring arranged between the activation button and the activation sleeve and configured to apply a biasing force on the activation sleeve,
wherein the drive mechanism is in a pre-tensioned and locked state, when the medicament delivery device is in a non-activated state, and
wherein the activation sleeve is configured to rotate due to an interaction of the inclined activation profile with the activation button when the activation button and the activation sleeve are moved towards each other in any order.

26. The subassembly of claim 25, wherein the plunger rod blocking element, preferably at least one support arm of the plunger rod blocking element, is configured to releasably engage with a distal ledge of the plunger rod, such that a proximal movement of the plunger rod is blocked when the medicament delivery device is in the non-activated state.

27. The subassembly of claim 26, wherein due to the rotation of the activation sleeve, each of the at least one openings of the activation sleeve is rotated to a position in which it matches the circumferential geometry of each of the at least one support arm of the support member, thereby enabling a radial outward deflection of each of the at least one support arm in order to disengage each of the at least one support arm from the distal ledge of the plunger rod and activate the medicament delivery device.

28. The subassembly of claim 25, wherein each of the at least one support arm is configured to interact with the distal ledge of the plunger rod via a radially inwardly inclined surface that is inclined in a proximal direction, such that due to the force of the pre-tensioned drive spring, the distal ledge of the plunger rod deflects each of the at least one support arm radially outwardly, in order to unlock the plunger rod and activate the medicament delivery device.

29. The subassembly of claim 25, wherein the activation button comprises a guide hole along a longitudinal axis of the subassembly configured to axially guide a distal end of the plunger rod when the medicament delivery device is activated.

30. A medicament delivery device comprising a distal subassembly and a proximal subassembly, wherein the distal subassembly is the subassembly according to claim 25.

31. The medicament delivery device of claim 30, wherein the proximal subassembly comprises:
a proximal housing connectable to the housing of the distal subassembly;
a rotator coaxially connected to a distal inner part of the proximal housing and movable in relation to the proximal housing;
a displaceable needle cover partially arranged within the proximal housing;
a medicament container holder connected through the needle cover and the rotator to the proximal housing and configured to receive a medicament container; and
a needle cover spring disposed between the needle cover and the medicament container holder and configured to apply a biasing force on the needle cover,
wherein the needle cover is axially movable inside at least the proximal housing such that the distal end of the needle cover contacts the needle cover contact surface of the activation sleeve whereby the activation sleeve is distally displaced.

32. The medicament delivery device of claim 30, wherein the medicament delivery device is a single-dose disposable auto-injector.
